(19) United States
(12) Patent Application Publication
(10) Pub. No.: US 2006/0099172 A1
(43) Pub. Date: May 11, 2006

(54) Method of cancer screening; method of cancer treatment; method of diabetes treatment; method of multiple sclerosis treatment; method of prophylaxis and treatment of avian influenza

(76) Inventor: John R. Woodward, Dallas, TX (US)

Correspondence Address: Michael A. O'Neil;Michael A. O'Neil, P.C., Suite 820, 5949 Sherry Lane, Dallas, TX, 75225, US

(21) Appl. No.: 11/292,662

(22) Filed: Dec. 2, 2005

Related U.S. Application Data
(63) Continuation-in-part of application No. 11/032,399, filed on Jan. 10, 2005.
Application No. 11/032,399 is a continuation-in-part of application No. 11/003,293, filed on Dec. 3, 2004.
Application No. 11/003,293 is a continuation-in-part of application No. 10/946,213, filed on Sep. 21, 2004.

(51) Int. Cl. A61K 38/19; A61K 31/522; A61K 31/4745

(52) U.S. Cl. 424/85.1; 514/263.32; 514/291; 424/400

(57) ABSTRACT

A method of treating avian influenza comprising transdermally administering imiquimod cream and DHEA cream while simultaneously administering valacyclovir tablets.

### CROSS-REFERENCE TO RELATED APPLICATIONS

This application is a continuation-in-part application of application Ser. No. 11/032,399 filed Jan. 10, 2005, currently pending, the entire contents of which are incorporated herein by reference; which is a continuation-in-part application of application Ser. No. 11/003,293 filed Dec. 3, 2004, currently pending, the entire contents of which are incorporated herein by reference; which is a continuation-in-part application of application Ser. No. 10/946,213 filed Sep. 21, 2004, currently pending, the entire contents of which are incorporated herein by reference.

### TECHNICAL FIELD

This invention relates generally to the detection and treatment of cancers, and more particularly to a method of screening for and a method of treating duct cell cancer of the breast, squamous cell cancer of the uterine cervix, anal cancer, diabetes, and multiple sclerosis. The invention further comprises a method of preventing and treatment avian influenza.

### BACKGROUND AND SUMMARY OF THE INVENTION

As is well known, various technologies are available to the medical profession for use in determining the presence of cancers in patients. Included are x-ray studies, magnetic resonance imaging (MRI) studies, CT scans, as well as studies of various body fluids such as blood, urine, etc. Potential sites for colon cancer, for example, can be investigated utilizing electro-optical technologies. In some cases needle biopsy or exploratory surgery is necessary to confirm either the presence or absence of suspected cancer.

Various techniques for treating cancers are also well known. Certain cancers can be surgically removed, whereas other cancers require radiation therapy, chemotherapy, or combinations of radiation therapy and chemotherapy. Other cancers are susceptible to control using one or more drug therapies.

Type I diabetes is generally diagnosed in juveniles and young adults. In type I diabetes, the pancreas does not make insulin, which is necessary for the body to process sugars. Persons with Type I diabetes can live long, healthy lives, but must be careful with their diet and must take insulin to manage their blood glucose levels. Currently, the only treatment for Type I diabetes is to take insulin, or receive pancreas or islet cell transplants.

Multiple sclerosis (MS) is a chronic, unpredictable disease of the nervous system that afflicts over 2.5 million persons worldwide. An MS attack destroys myelin, the protective fibers around nerve fibers in the central nervous system. The destroyed myelin is replaced by scars of hardened “sclerotic” tissue, and some nerve endings are permanently severed. The common symptoms are loss of balance, fatigue, poor circulation, slurred speech, blindness, and in some cases paralysis. Currently, the only treatment is disease-modifying drugs, including drugs with a chemotherapeutic agent. These treatments only modify the disease to lessen the severity or frequency of the MS attacks.

At the present time outbreaks of avian influenza are occurring around the world, particularly in Asia. The possibility of an avian influenza pandemic is well recognized, as is the fact that an avian influenza pandemic would kill millions of people worldwide. At the present time there does not exist an effective method of preventing and treatment avian influenza.

The present invention comprises a method of cancer screening, a method of cancer treatment, a method for treatment of diabetes, and a method for treatment of multiple sclerosis which has proven successful in controlling epidermal cancers including, but not limited to, duct cell breast cancer, cervical squamous cancer, and anal cancer, controlling Type I diabetes, and controlling multiple sclerosis. In accordance with the broader aspects of the invention, a method of cancer screening involves a series of testing procedures each more expensive than the one before. Only when results of each of the testing procedures are positive is the presence of cancer confirmed. The invention further comprises a method of treating cancer and insulin dependent Type I diabetes wherein the drug imiquimod is administered transdermally in conjunction with a vaccine containing tumor necrosis factor alpha, for example the BCG vaccine, and valacyclovia hydrochlorine tablets. The method of the present invention treats Type I diabetes by enabling the body to regenerate islet cells. Similarly, multiple sclerosis is managed and treated by enabling the body to repair nerve endings and regenerate damaged fibers.

Another embodiment of the invention comprises a method of prophylaxis and treatment of avian influenza. In accordance therewith avian influenza a.k.a. bird flu is prevented by daily transdermal administrations of dehydroepiandrosterone (DHEA) and interferon alpha together with VALTREX® (valacyclovir) taken orally. Avian influenza infections are treated similarly except the daily dosage of DHEA is substantially increased.

### BRIEF DESCRIPTION OF THE DRAWING

A more complete understanding of the present invention may be had by reference to the following Detailed Description when taken in connection with the accompanying Drawings, wherein:

FIG. 1 is a flowchart illustrating initial steps in the cancer screening method of the present invention;

FIG. 2 is a flowchart illustrating subsequent steps in the cancer screening method of the present invention; and

FIG. 3 is a flowchart illustrating the diabetes treatment method of the present invention.

### DETAILED DESCRIPTION

### Introduction

The following examples describe a method of detecting and treating duct cell breast cancer, and a method for treating Type I diabetes. However, the present invention is equally applicable to other epidermal cancers, such as squamous cancer of the uterine cervix and anal cancer, and the treatment of and management of multiple sclerosis.

### EXAMPLE

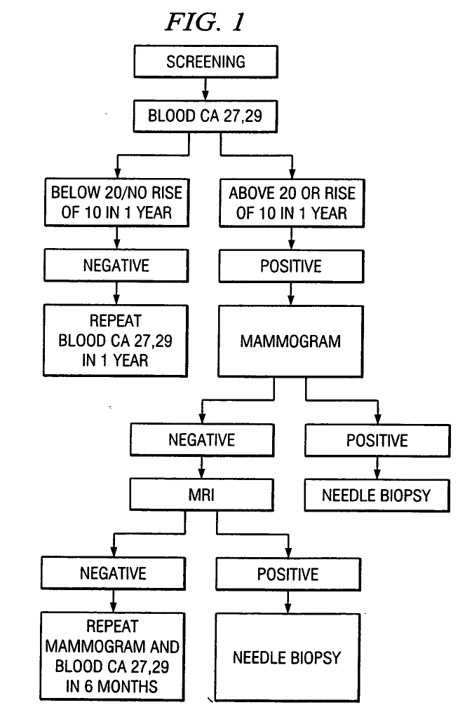

Referring to the Drawings, and particularly to FIG. 1 thereof, the early steps in the method of cancer screening of the present invention are shown therein. Screening begins with administration of the testing procedure known as Blood CA 27,29. The Blood CA 27,29 testing procedure has heretofore been utilized in monitoring the results of existing cancer treatment procedures. However, the Blood CA 27,29 procedure has not heretofore been used for cancer screening.

If the number comprising the results of the Blood CA 27,29 procedure is less than 20, and if there has been no increase in the number comprising the result of the Blood CA 27,29 testing procedure of ten (10) or more in the immediately preceding year, the result of the Blood CA 27,29 testing procedure is considered to be negative. The patient is then scheduled for follow-up testing utilizing the Blood CA 27,29 procedure in one year.

If the number comprising the result of the Blood CA 27,29 procedure is 20 or above, or if there has been an increase of 10 or more in the number comprising the result of the CA 27,29 testing procedure in the immediately preceding year, the result of the Blood CA 27,29 procedure is considered to be positive. In that event a mammogram testing procedure is administered. If the result of the mammogram testing procedure is negative, an MRI testing procedure is administered. If the result of the MRI testing procedure is negative, both the mammogram testing procedure and the Blood CA 27,29 testing procedure are re-administered in six months time. Conversely, if either the mammogram testing procedure is positive or the MRI testing procedure is positive, a needle biopsy of the identified lesion is performed.

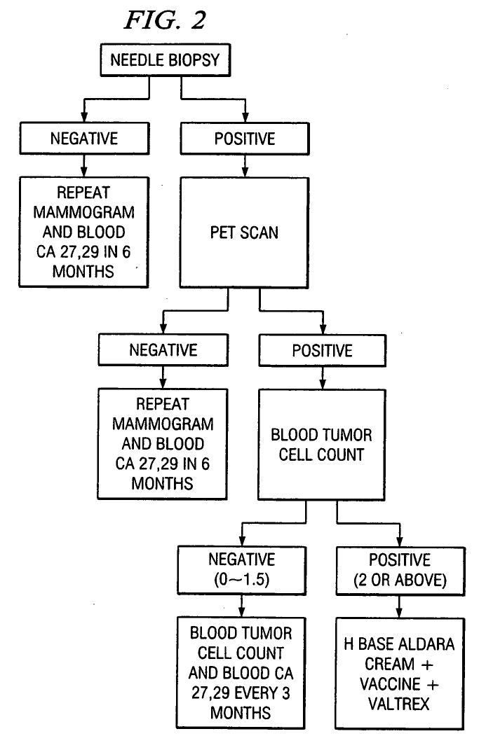

Referring to FIG. 2, if the results of the needle biopsy testing procedure are negative, both the mammogram testing procedure and the Blood CA 27,29 testing procedure are re-administered in six months. If the needle biopsy testing procedure is positive, a positron emission tomography (PET) scan testing procedure is administered.

The cancer treatment procedure of the present invention comprises the transdermal administration of the drug imiquimod combined with a vaccine containing tumor necrosis factor alpha and administration of valacyclovia hydrochlorine tablets. Imiquimod is commercially available from 3M Pharmaceuticals under the trademark ALDARA® (imiquimod). A healthy human body produces the protein interferon alpha in response to an infection. Interferon alpha works to coat the infection or virus in order to make the infection or virus vulnerable to the human immune system. Imiquimod cream turns into interferon alpha once inside the human body, providing the needed interferon alpha not adequately produced by the patient's own body. In accordance with the present invention, ALDARA® (imiquimod) cream 5% is mixed at a 1:1 ratio with H base cream. The ingredients of H base cream are:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.
   The mixture of imiquimod and H base cream as described above is administered transdermally, preferably by mixing ¼ cc ALDARA® (imiquimod) 5% cream with ¼ cc H base cream and applying the resulting mixture to various locations, i.e., the inner thigh, abdomen, hip, arms, etc., of the patient. Various sites of administration prevent any possible skin irritation. The foregoing amount of the mixture of ALDARA® (imiquimod) 5% cream and H base cream is applied daily.

Tumor necrosis factor alpha (also called TNFa, cachexin, or cachetin) stimulates T-cells that coordinate the immune system. In a healthy human body, tumor necrosis factor alpha is released by white blood cells and other tissues in response to damage caused by an infection. Tumor necrosis factor alpha is found in several vaccines. The preferable vaccine to be used in accordance with the present invention is the BCG vaccine, which is a common vaccine for tuberculosis, given in the United States and around the world. The tumor necrosis factor alpha included in the BCG vaccine and imiquimod cream work together to stimulate the T-cells and coordinate and improve the patient's immune system. Interferon alpha coats the virus or infection in order to make the virus or infection vulnerable to the tumor necrosis factor alpha.

In accordance with the present invention, the vaccine is given in doses of 0.1 mL (100 μg) once every three weeks as long as the treatment continues. Valacyclovia hydrochlorine tablets, available from GlaxoSmithKline under the trademark VALTREX® (valacyclovir) is drug commonly used in the treatment for genital herpes. In accordance with the present invention, VALTREX® (valacyclovir) tablets are consumed twice daily in 500 mg doses. The combination of the ALDARA® (imiquimod) and H base cream, the BCG vaccination, and the VALTREX® (valacyclovir) tablets is administered until a blood tumor cell count indicates that there are no cancer cells in the blood and a subsequent blood tumor cell count verifies a normal cell count and no mestastases are present. The results of the procedure are periodically monitored utilizing the Blood CA 27,29 testing procedure.

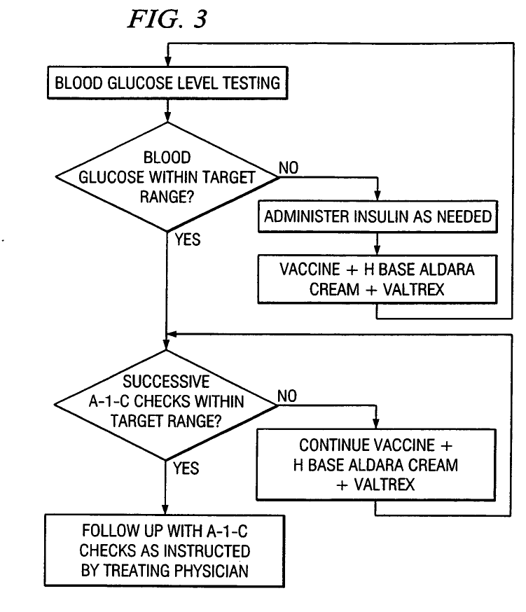

Referring to FIG. 3 thereof, persons with Type I diabetes must check their blood glucose levels at multiple intervals as directed by their physician. The average fasting blood glucose level should be between 70 mg/dL and 110 mg/dL. If the blood glucose level is not within the target level, the level must be corrected by taking insulin.

The diabetes treatment procedure of the present invention comprises the transdermal administration of the drug imiquimod combined with a BCG vaccination and administration of valacyclovia hydrochlorine tablets. Imiquimod is commercially available from 3M Pharmaceuticals under the trademark ALDARA® (imiquimod). A healthy human body produces the protein interferon alpha in response to an infection. Interferon alpha works to coat the infection or virus in order to make the infection or virus vulnerable to the human immune system. Imiquimod cream turns into interferon alpha once inside the human body, providing the needed interferon alpha not adequately produced by the patient's own body.

In accordance with the present invention, ALDARA® (imiquimod) cream 5% is mixed at a 1:1 ratio with H base cream. The ingredients of H base cream are:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.
   The mixture of imiquimod and H base cream as described above is administered transdermally, preferably by mixing ¼ cc ALDARA® (imiquimod) 5% cream with ¼ cc H base cream and applying the resulting mixture to various locations, i.e., the inner thigh, abdomen, hip, arms, etc., of the patient. Various sites of administration prevent any possible skin irritation. The foregoing amount of the mixture of ALDARA® (imiquimod) 5% cream and H base cream is applied daily.
   Tumor necrosis factor alpha (also called TNFa, cachexin, or cachetin) stimulates T-cells that coordinate the immune system. In a healthy human body, tumor necrosis factor alpha is released by white blood cells and other tissues in response to damage caused by an infection. Tumor necrosis factor alpha is found in several vaccines. The preferable vaccine to be used in accordance with the present invention is the BCG vaccine, which is a common vaccine for tuberculosis, given in the United States and around the world. The tumor necrosis factor alpha included in the BCG vaccine and imiquimod cream, work together to stimulate the T-cells and coordinate and improve the patient's immune system. Interferon alpha coats the virus or infection in order to make the virus or infection vulnerable to the tumor necrosis factor alpha.

In accordance with the present invention, the vaccine is given in doses of 0.1 mL (100 μg) once every three weeks as long as the treatment continues. Valacyclovia hydrochlorine tablets, available from GlaxoSmithKline under the trademark VALTREX® (valacyclovir) is drug commonly used in the treatment for genital herpes. In accordance with the present invention, VALTREX® (valacyclovir) tablets are consumed twice daily in 500 mg doses.

The results of the procedure are monitored utilizing blood sugar meters and a diary to record ongoing blood sugar levels. Additionally, A-1-C checks track the patient's overall blood sugar levels over two to three month periods, and is the most effective way to track long-range success of the treatment. The treatment method regenerates islet cells, which produce insulin. Once the patient no longer depends on insulin to correct blood sugar levels, the treatment continues until the patient has two or more sequential A-1-C checks in the target range, depending on the judgment of the treating physician. Once treatment is discontinued, A-1-C checks continue, but at less frequent intervals as recommended by the treating physician.

The present invention further comprises a method of treatment multiple sclerosis (MS). In accordance with the invention the treatment regiment for MS is the same as the treatment regimens described above in conjunction with cancer treatment and diabetes treatment.

The avian influenza prevention and treatment procedures of the present invention comprise the transdermal administration of the drug imiquimod combined with a substance containing tumor necrosis factor alpha and administration of valacyclovia hydrochlorine tablets. Imiquimod is commercially available from 3M Pharmaceuticals under the trademark ALDARA® (imiquimod). A healthy human body produces the protein interferon alpha in response to an infection. Interferon alpha works to coat the infection or virus in order to make the infection or virus vulnerable to the human immune system. Imiquimod cream turns into interferon alpha once inside the human body, providing the needed interferon alpha not adequately produced by the patient's own body.

In accordance with the present invention, ALDARA® (imiquimod) cream 5% is mixed at a 1:1 ratio with H base cream. The ingredients of H base cream are:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.
   The mixture of imiquimod and H base cream as described above is administered transdermally, preferably by mixing ½ cc ALDARA® (imiquimod) 5% cream with ½ cc H base cream and applying the resulting mixture to various locations, i.e., the inner thigh, abdomen, hip, arms, etc., of the patient. Various sites of administration prevent any possible skin irritation. The foregoing amount of the mixture of ALDARA® (imiquimod) 5% cream and H base cream is applied daily.

Tumor necrosis factor alpha (also called TNFa, cachexin, or cachetin) stimulates T-cells that coordinate the immune system. In a healthy human body, tumor necrosis factor alpha is released by white blood cells and other tissues in response to damage caused by an infection. Tumor necrosis factor alpha is found in several vaccines. The preferable source of tumor necrosis factor alpha is dehydroepiandrosterone (DHEA). Preferably, between about ½ cc and 1 cc of DHEA cream (concentration: ______%) is mixed with a corresponding amount of the imiquimod mixture described in the preceding paragraph hereof. DHEA cream is available over the counter and can also be compounded by any licensed pharmacist.

The tumor necrosis factor alpha included in the DHEA vaccine and imiquimod cream work together to stimulate the T-cells and coordinate and improve the patient's immune system. Interferon alpha coats the virus or infection in order to make the virus or infection vulnerable to the tumor necrosis factor alpha.

Valacyclovia hydrochlorine tablets, available from GlaxoSmithKline under the trademark VALTREX® (valacyclovir) is a drug commonly used in the treatment for genital herpes. In accordance with the present invention, VALTREX® (valacyclovir) tablets are consumed once daily in 500 mg doses. The combination of the ALDARA® (imiquimod), H base cream, DHEA, and the VALTREX® (valacyclovir) tablets is administered so long as a risk of avian influenza infection persists. If an actual infection of avian influenza is diagnosed, the procedure continues except that the administration amount of DHEA is quadrupled.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of preventing infections of avian influenza comprising the steps of:
providing a quantity of ALDARA® (imiquimod) 5% cream;
providing a quantity of H base cream;
mixing ½ cc. of the ALDARA® (imiquimod) 5% cream with ½ cc. of the H base cream and transdermally administering the resulting mixture to the patient;
simultaneously transdermally administering tumor necrosis factor alpha to the patient; and
orally administering VALTREX® (valacyclovir) tablets.
